(19) United States
(12) Patent Application Publication
(10) Pub. No.: US 2007/0275978 A1
(43) Pub. Date: Nov. 29, 2007

(54) Of at Use of an Antagonist Compound of at Least One Receptor Selected From a Group Comprising Beta-Adrenergic Receptors, A at1, 5-Ht5 and Galanin Receptor for Preparing a Pharmaceutical Composition for Treating Rosacea

(75) Inventors: Fabrizio Dolfi, Valbonne (FR); Irina Safonova, Nice (FR)

Correspondence Address: OBLON, SPIVAK, MCCLELLAND MAIER & NEUSTADT, P.C., 1940 DUKE STREET, ALEXANDRIA, VA, 22314, US

(73) Assignee: Galderma Research & Development S.N.C., Valbonne Sophia Antipolis (FR)

(21) Appl. No.: 10/590,077

(22) PCT Filed: Feb. 17, 2005
(86) PCT No.: PCT/FR05/00367

§ 371 Date: Jul. 18, 2007

(30) Foreign Application Priority Data
Feb. 20, 2004 (FR) 04/01715

(51) Int. Cl. A61K 31/195; A61K 31/405; A61K 31/41; A61K 31/497; A61K 31/517; A61K 31/519; A61P 17/10

(52) U.S. Cl. 514/254.06; 514/259.41; 514/266.22; 514/362; 514/381; 514/394; 514/397; 514/415; 514/563; 514/567

(57) ABSTRACT

The invention relates to the use of an antagonist of at least one receptor chosen from the group comprising the beta-adrenergic receptors, the AT1 receptor, the 5-HT2 receptor, the 5-HT5 receptor and the galanin receptor, for the preparation of a pharmaceutical composition for treating rosacea.

The present invention relates to the field of treating rosacea. The invention is directed towards providing novel pharmaceutical compositions, more particularly dermatological compositions, which are useful for treating rosacea and comprising as active agent an antagonist of at least one receptor chosen from the group comprising the beta-adrenergic receptors, the AT1 receptor, the 5-HT2 receptor, the 5-HT5 receptor and the galanin receptor.

Rosacea is a common, chronic and progressive inflammatory dermatitis associated with vascular instability. It mainly affects the central part of the face and is characterized by redness of the face or hot flushes, facial erythema, papules, pustules and telangiectasia. In serious cases, especially in men, the soft tissue of the nose may swell and produce a bulbous swelling known as rhinophyma.

Rosacea generally occurs between the ages of 25 and 70, and is much more common in people of fair complexion. It more particularly affects women, although this affection is generally more severe in men. Rosacea is chronic and lasts for years with periods of exacerbation and of remission.

Rosacea was originally called “acne rosacea” because its papules (points of slight raising of the skin) and its inflammatory pustules (pus scabs) greatly resemble those of common acne. In contrast with common acne, whose aetiology is based on abnormal keratinization, an increase in sebum production and also bacterial inflammation, the inflammation of rosacea is vascular in nature and is poorly understood. The result of this facial vascular anomaly is a permanent oedema of the dermis, which may be accompanied by an increased colonization with Demodex folliculorum, a mite usually found in the follicles of the face.

According to various studies, Demodex folliculorum is thought to have an aetiological role in rosacea (Erbagi et al. 1998, Int. J. Dermatol., vol. 37, pages 421-425; Purcell et al. 1986, J. Am. Acad. Dermatol., vol. 15, pages 1159-1162; Sibenge et al. 1992, J. Am. Acad. Dermatol., vol. 26, pages 590-593). It appears that Demodex folliculorum causes or aggravates inflammatory reactions, reflected by papules and pustules, by blocking the pilosebaceous follicles of the face (Roihu et al. 1998, J. Cutan. Pathol., vol. 25, pages 550-552). This parasite is moreover thought to trigger a humoral immune response (Nunzi et al. 1980, Br. J. Dermatol., vol. 103, pages 543-551; Manna et al. 1982, Br. J. Dermatol., vol. 107, pages 203-208).

The pathogenesis of rosacea is poorly understood. Many factors may be involved without necessarily inducing this complaint. They are, for example, psychological factors, gastrointestinal disorders, environmental factors (exposure to sunlight, temperature, humidity), emotional factors (stress), dietary factors (alcohol, spices), hormonal factors or vascular factors, or even infection with Helicobacter pilori.

Rosacea develops in four stages, but passage through all the stages is not obligatory:

stage 1 of vascular relaxation (at about 20 years old). The patients have sudden bursts of paroxystic redness of the face and neck, with a hot sensation, but with no systemic signs. After the attacks, the skin of the face returns to normal. These “flushes” are triggered by changes in temperature (occasionally leading to thermophobia), and the intake of hot drinks or alcohol;

stage 2 of erythemato-telangiectasia (at about 30 years old). The cheekbone areas are diffusely red. Dilated capillaries constituting standard acne rosacea are observed therein. In contrast with stage 1, the redness is permanent. Besides the cheeks, the chin and the middle of the forehead may be affected;

stage 3 of papulo-pustules (at about 40 years old). Papules and pustules a few millimetres in diameter develop on a background of erythema, without associated comedones. This dermatitis may be very extensive, occasionally up to the bald part of the scalp in men, but is absent from the area around the mouth and the eyes. The patients complain of sensitive skin, with subjective intolerance to the majority of topical products and greasy cosmetics;

stage 4 of rhinophyma (at about 50 years old or later). This late phase mainly affects men, in contrast with the other stages. The nose is increased in volume and diffusely red, and the follicular orifices are dilated. The skin gradually thickens.

The minor forms of rosacea may be treated with active agents such as anti-seborrhoeic agents and anti-infectious agents, for example benzoyl peroxide, retinoic acid or metronidazole. Metronidazole, or (2-methyl-5-nitroimidazolyl)-2-ethanol, is known in the prior art for its antibacterial, anti-parasitic and anti-protozoan properties. It exerts selective toxicity towards anaerobic microorganisms and also hypoxic cells. In the latter, metronidazole is reduced to derivatives capable of impairing the DNA structure of these cells.

As regards the most diffuse forms of the complaint, they respond well to general antibiotic therapy with cyclines. However, these treatments have unpleasant side effects for the patient, such as irritation or intolerance phenomena.

Furthermore, on account of the multi-factor aspect of rosacea, there are a huge number of treatments for this condition, but the search continues for an effective treatment that is without risk for the patient.

The Applicant's studies have demonstrated the usefulness of antagonists of receptors chosen from the group comprising the beta-adrenergic receptors, the AT1 receptor, the 5-HT2 receptor, the 5-HT5 receptor and the galanin receptor in the treatment of rosacea.

The beta-adrenergic receptors are involved in regulating various physiological functions, such as metabolic activity, cardiac activity, respiration, central nervous system activity, the blood pressure and the vascular tonus.

The 5-HT2 receptors and the 5-HT5 receptors belong to the family of serotonin (5-HT) receptors. The 5-HT receptors are all coupled to G proteins, except for 5-HT3, which is an ion channel. Activation of the 5-HT2 receptors stimulates the activity of phospholipase C. The 5-HT5 receptor transduction system is positively associated with adenylate cyclase.

The AT1 receptor is involved in regulating vasoconstriction by angiotensin II. In man, angiotensin II increases the tonus of the subcutaneous arteries.

Galanin is a 29-amino-acid peptide present in the central nervous system. According to certain studies, galanin is thought to play a role in modulating the cutaneous vascular reaction and in inflammation (Pincelli, 1990, Br. J. Dermatol., vol. 122, pages 745-750).

The Applicant's studies have demonstrated the involvement of antagonists of the beta-adrenergic receptors, the AT1 receptor, the 5-HT2 receptor, the 5-HT5 receptor and the galanin receptor in the treatment of rosacea. This activity was demonstrated by using metronidazole, which has the consequence of interacting with the beta-adrenergic receptors! the AT1 receptor, the 5-HT2 receptor, the 5-HT5 receptor and the galanin receptor. More particularly, it has been found that the use of metronidazole inhibits the binding of the natural ligands to these receptors.

As indicated previously, the invention is directed towards offering a novel method for treating rosacea, which consists in administering to an individual suffering from rosacea an effective amount of an antagonist of at least one receptor chosen from the group comprising the beta-adrenergic receptors, the ATl receptor, the 5-HT2 receptor, the 5-HT5 receptor and the galanin receptor.

Consequently, the invention relates more particularly to the use of an antagonist of at least one receptor chosen from the group comprising the beta-adrenergic receptors, the ATl receptor, the 5-HT2 receptor, the 5-HT5 receptor and the galanin receptor, for the preparation of a pharmaceutical composition for treating rosacea.

The invention also relates to the use of an antagonist of two receptors chosen from the group comprising the beta-adrenergic receptors, the ATl receptor, the 5-HT2 receptor, the 5-HT5 receptor and the galanin receptor, for the preparation of a pharmaceutical composition as defined above.

The invention also relates to the use of an antagonist of three receptors chosen from the group comprising the beta-adrenergic receptors, the ATl receptor, the 5-HT2 receptor, the 5-HT5 receptor and the galanin receptor, for the preparation of a pharmaceutical composition as defined above.

The invention also relates to the use of an antagonist of four receptors chosen from the group comprising the beta-adrenergic receptors, the AT1 receptor, the 5-HT2 receptor, the 5-HT5 receptor and the galanin receptor, for the preparation of a pharmaceutical composition as defined above.

The invention also relates to the use of an antagonist of five receptors chosen from the group comprising the beta-adrenergic receptors, the AT1 receptor, the 5-HT2 receptor, the 5-HT5 receptor and the galanin receptor, for the preparation of a pharmaceutical composition as defined above.

According to the present invention, the term “antagonist” means any molecule that inhibits the binding of at least one agonist or a natural ligand to its receptor.

Non-limiting examples of antagonists of the beta-adrenergic receptors that may be mentioned include metropobol, esmolol, acebutolol, timolol, pindolol and labetolol.

Non-limiting examples of antagonists of the AT1 receptor that may be mentioned include candesartan, cilexil, losartan, irbesartan, telmisartan, valsartan and eprosartan.

Non-limiting examples of antagonists of the 5-HT2 and 5-HT5 receptors that may be mentioned include ketanserin, trazodone and risperidone.

More particularly, the composition that is the subject of the present invention is a dermatological composition for topical administration to the skin.

According to the present invention, the term “treating rosacea” means the treatment and/or prevention of rosacea, at one or more of the stages described above.

According to a first embodiment of the invention. the composition is intended for treating the first stage of rosacea.

According to a second embodiment of the invention, the composition is intended for treating the second stage of rosacea.

According to a third embodiment of the invention, the composition is intended for treating the third stage of rosacea.

According to a fourth embodiment of the invention, the composition is intended for treating the fourth stage of rosacea.

According to a preferential embodiment, the composition contains from 0.0001% to 20% by weight, preferably from 0.1% to 2% and more preferentially from about 0.75% to 1% of an antagonist as defined above.

Needless to say, the present invention concerns, besides the use of an antagonist as defined above, the use of derivatives thereof. The term “derivatives” means compounds that differ from the antagonist as defined above by substitution, addition or removal of one or more chemical groups.

Advantageously, the compositions of the invention comprise, besides the antagonist as defined above, at least one other therapeutic agent capable of increasing the efficacy of the treatment. Non-limiting examples of such agents that may be mentioned include antibiotics, antibacterial agents, antiviral agents, antiparasitic agents, antifungal agents, anaesthetics, analgesics, antiallergic agents, retinoids, free-radical scavengers, anti-pruriginous agents, keratolytic agents, anti-seborrhoeic agents, antihistamines, sulfides, immunosuppressant products and anti-proliferative products.

According to one particular embodiment of the invention, the antagonist is not metronidazole. According to another particular embodiment of the invention, the composition of the present invention also contains metronidazole.

The invention also relates to a process for identifying an antagonist of at least one receptor chosen from the group comprising the beta-adrenergic receptors, the AT1 receptor, the 5-HT2 receptor, the 5-HT5 receptor and the galanin receptor:

a) placing the radiolabelled specific ligand in contact with the human recombinant protein of each receptor in a sample;

b) placing the radiolabelled specific ligand and the non-radiolabelled specific ligand in excess in contact with the human recombinant protein of each receptor in another sample;

c) adding the test compound to the two samples;

d) measuring the radioactivity by scintillation counting in the two samples;

e) calculating the difference in radioactivity measured in the two samples;

f) selecting the said compounds for which a reduction in radioactivity is obtained in step e) relative to the control value obtained with the receptors not placed in contact with the test compound.

The compositions of the invention may also comprise any additive usually used in the pharmaceutical or dermatological field that is compatible with the antagonist as defined above. Mention may be made especially of sequestrants, antioxidants, sunscreens, preserving agents, for example DL-α-tocopherol, fillers, electrolytes, humectants, dyes, common mineral or organic acids or bases, fragrances, essential oils, cosmetic active agents, moisturizers, vitamins, essential fatty acids, sphingolipids, self-tanning compounds such as DHA, skin calmative and protective agents such as allantoin, pro-penetrating agents and gelling agents. Needless to say, a person skilled in the art will take care to select this or these optional additional compound(s), and/or the amount thereof, such that the advantageous properties of the composition according to the invention are not, or are not substantially, adversely affected.

These additives may be present in the composition in a proportion of from 0 to 20% by weight relative to the total weight of the composition.

Examples of sequestrants that may be mentioned include ethylenediaminetetraacetic acid (EDTA), and also derivatives or salts thereof, dihydroxyethylglycine, citric acid and tartaric acid, or mixtures thereof.

Examples of preserving agents that may be mentioned include benzalkonium chloride, phenoxyethanol, benzyl alcohol, diazolidinylurea and parabens, or mixtures thereof.

Examples of humectants that may be mentioned include glycerol and sorbitol.

The compositions of the invention may contain one or more pro-penetrating agents in preferential concentrations ranging from 0 to 20% and more preferentially ranging from 0.6% to 3% by weight relative to the total weight of the composition. Among the pro-penetrating agents that are preferentially used, without this list being limiting, are compounds such as propylene glycol, dipropylene glycol, propylene glycol dipelargonate, lauroglycol and ethoxydiglycol.

Advantageously, the compositions according to the invention may also contain one or more wetting liquid surfactants in preferential concentrations ranging from 0 to 10% and more preferentially ranging from 0.1% to 2%.

The compositions of the present invention may be in any galenical form normally used for topical application, especially in the form of aqueous, aqueous-alcoholic or oily solutions, dispersions of the lotion type, aqueous, anhydrous or lipophilic gels, emulsions of liquid or semi-liquid consistency of the milk type, obtained by dispersing a fatty phase in an aqueous phase (O/W) or, conversely, (W/O), or suspensions or emulsions of soft, semi-solid or solid consistency of the cream, gel or ointment type, or alternatively microemulsions, microcapsules, microparticles or vesicular dispersions of ionic and/or nonionic type.

Preferably, the creams may be formulated from a mixture of mineral oil or from a mixture of beeswax and of water, which emulsifies instantaneously, to which is added the antagonist as defined above, dissolved in a small amount of oil such as almond oil.

The ointments may be formulated by mixing a solution of the said antagonist in an oil such as almond oil in warmed paraffin, followed by leaving the mixture to cool.

As examples of compositions according to the invention, mention may be made of those comprising an active phase containing (expressed as weight percentages):

0 to 90%, preferentially 5% to 25% and especially 10% to 20% of water;

0 to 10%, preferentially 0 to 2% and especially 0 to 0.5% of wetting liquid surfactant;

0 to 20%, preferentially 0 to 10% and especially 2% to 5% of pro-penetrating agent;

0.0001% to 20% and preferentially 0.1% to 2% of the said antagonist;

and an aqueous phase comprising a pH-independent gelling agent, and water.

The aqueous phase of a composition according to the invention in the form of an emulsion may comprise water, a floral water such as cornflower water or a natural spring or mineral water chosen, for example, from eau de Vittel, the waters of the Vichy basin, eau d'Uriage, eau de la Roche Posay, eau de la Bourboule, eau d'Enghien-les-Bains, eau de Saint Gervais-les-Bains, eau de Néris-les-Bains, eau d'Allevard-les-Bains, eau de Digne, eau de Maizières, eau de Neyrac-les-Bains, eau de Lons-le-Saunier, les Eaux Bonnes, eau de Rochefort, eau de Saint Christau, eau des Fumades, eau de Tercis-les-Bains, eau d'Avène and eau d'Aix-les-Bains.

The said aqueous phase may be present in a content of between 10% and 90% by weight and preferably between 20% and 80% by weight relative to the total weight of the composition.

Non-limiting examples that may be mentioned include gelling agents of the polyacrylamide family such as the sodium acryloyldimethyltaurate copolymer/isohexadecane/polysorbate-80 mixture sold under the name Simulgel 600 by the company SEPPIC, the polyacrylamide/C13-14 isoparaffin/laureth-7 mixture, for instance the product sold under the name Sepigel 305 by the company SEPPIC, the family of acrylic polymers coupled to hydrophobic chains, such as the PEG-150/decyl/SMDI copolymer sold under the name Aculyn 44 (polycondensate comprising at least, as components, a polyethylene glycol containing 150 or 180 mol of ethylene oxide, decyl alcohol and methylenebis(4-cyclohexyl isocyanate) (SMDI), at 35% by weight in a mixture of propylene glycol (39%) and water (26%)), and the family of modified starches such as the modified potato starch sold under the name Structure Solanace, or mixtures thereof.

The preferred gelling agents are derived from the polyacrylamide family, such as Simulgel 600 or Sepigel 305 or mixtures thereof.

The gelling agent as described above may be used in preferential concentrations ranging from 0.1% to 15% and more preferentially ranging from 0.5% to 5%.

The gels may preferably be prepared by dispersing or dissolving metronidazole in a suitable ratio in a gel of carbomer, poloxamer or cellulose-based type.

Other advantages and characteristics of the invention will emerge from the examples below concerning the activity of metronidazole as an antagonist of receptors chosen from the group comprising the beta-adrenergic receptors, the AT1 receptor, the 5-HT2 receptor, the 5-HT5 receptor and the galanin receptor.

### Example 1

### Measurement of the Binding to the Beta-1 and Beta-2 Adrenergic, AT1, 5-HT₅A, 5-HT2A and Galanin Receptors

1) Protocol:

The test of binding to the beta-1 and beta-2 adrenergic receptors was performed according to the method described by Smith and Teiler 1999, Cardiovasc. Drug Ther., vol. 13, pages 123-126.

The test of binding to the AT1 receptor was performed according to the method described by Bergsma et al. 1992, Biochem. Biophys. Res. Comm., vol. 183, pages 989-995.

The test of binding to the 5-HT_{5A} receptor was performed according to the method described by Ress et al. 1994, FEBS Lett., vol. 355, pages 242-246.

The test of binding to the 5-HT_{2A} receptor was performed according to the method described by Bonhauss et al. 1995, Brit. J. Pharmacol., vol. 1155, pages 622-628.

The test of binding to the galanin receptor was performed according to the method described by Sullivan et al. 1997, Biochem. Biophys. Res. Comm., vol. 233, pages 823-828.

2) Experimental Conditions:

The binding of metronidazole to each receptor was determined by competitive experiments. The receptor, human recombinant protein, was incubated for times indicated in Table 1 below, with a simple concentration of labelled specific ligand, in the presence of 10 μM metronidazole. The bound radioactivity was measured by scintillation counting.

TABLE 1

| | Radiolabelled | | Incubation |
|---|---|---|---|
| Receptor | specific ligand | Non-specific ligand | conditions |
| Beta₁ | [³H]CGP 12177 | Aprenolol (50 μM) | 60 min/22° C. |
| adrenergic | (0.15 nM) | | |
| Beta₂ | [³H]CGP 12177 | Aprenolol (50 μM) | 60 min/22° C. |
| adrenergic | (0.15 nM) | | |
| AT1 | [¹²⁵I][Sar¹Ile⁸] | Angiotensin II | 60 min/22° C. |
| | AII (0.05 nM) | (10 μM) | |
| 5-HT2A | [³H]ketanserin | Ketanserin (1 μM) | 15 min/37° C. |
| | (0.5 nM) | | |
| 5-HT5A | [³H]LSD (1 nM) | Serotonin (100 μM) | 30 min/37° C. |
| Galanin 1 | [¹²⁵I]galanin | Galanin (1 μM) | 60 min/22° C. |
| | (0.03 nM) | | |

3) Analysis and Expression of the Results:

The specific binding of the ligand to the receptor is defined as the difference between the total binding and the non-specific binding determined in the presence of an excess of unlabelled ligand.

The results are given in Table 2 below, expressed as a percentage of control specific binding and as a percentage of inhibition of the control specific binding obtained in the presence of metronidazole.

TABLE 2

| | | % of control specific |
|---|---|---|
| Receptor | Metronidazole (μM) | binding (±SD) |
| Beta₁ adrenergic | 10 | 85.1 ± 1.9 |
| Beta₂ adrenergic | 10 | 85.9 ± 4.3 |
| ATI | 10 | 79.7 ± 2.0 |
| 5-HT2A | 10 | 81.6 ± 0.8 |
| 5-HT5A | 10 | 83.2 ± 3.1 |
| Galanin 1 | 10 | 81.1 ± 3.4 |

Metronidazole thus inhibits the binding to the beta-adrenergic receptors, to the AT1 receptor, to the 5-HT2 receptor, to the 5-HT5 receptor and to the galanin receptor.

1. A pharmaceutical composition for treating rosacea, comprising a rosacea treating effective amount of an antagonist of at least one receptor selected from the group consisting of the beta-adrenergic receptors, the AT1 receptor, the 5-HT2 receptor, the 5-HT5 receptor and the galanin receptor, with the proviso that the antagonist is not metronidazole.

2. The composition according to claim 1, wherein the antagonist is an antagonist of two receptors selected from the group consisting of the beta-adrenergic receptors, the AT1 receptor, the 5-HT2 receptor, the 5-HT5 receptor and the galanin receptor.

3. The composition according to claim 1, wherein the antagonist is an antagonist of three receptors selected from the group consisting of the beta-adrenergic receptors, the AT1 receptor, the 5-HT2 receptor, the 5-HT5 receptor and the galanin receptor.

4. The composition according to claim 1, wherein the antagonist is an antagonist of four receptors selected from the group consisting of the beta-adrenergic receptors, the AT1 receptor, the 5-HT2 receptor, the 5-HT5 receptor and the galanin receptor.

5. The composition according to claim 1, wherein the antagonist is an antagonist of five receptors selected from the group consisting of the beta-adrenergic receptors, the AT1 receptor, the 5-HT2 receptor, the 5-HT5 receptor and the galanin receptor.

6. The composition according to claim 1, wherein the antagonist is at least one antagonist of the beta-adrenergic receptors and selected from the group consisting of metropobol, esmolol, acebutolol, timolol, pindolol and labetolol.

7. The composition according to claim 1, wherein the antagonist is at least one antagonist of the AT1 receptor and selected from the group consisting of candesartan, cilexil, losartan, irbesartan, telmisartan, valsartan and eprosartan.

8. The composition according to claim 1, wherein the antagonist is at least one antagonist of the 5-HT2 and 5-HT5 receptors and selected from the group consisting of ketanserin, trazodone and risperidone.

9. The composition according to claim 1, wherein the composition is a dermatological composition for topical application.

10. The composition according to claim 1, wherein the composition is for treating at least one stage of rosacea.

11. The composition according to claim 1, wherein the composition is for treating the first stage of rosacea.

12. The composition according to claim 1, wherein the composition is for treating the second stage of rosacea.

13. The composition according to claim 1, wherein the composition is for treating the third stage of rosacea.

14. The composition according to claim 1, wherein the composition is for treating the fourth stage of rosacea.

15. The composition according to claim 1, wherein the composition contains from 0.0001% to 20% by weight the antagonist.

16. The composition according to claim 1, further comprising another active agent chosen from the group of antibiotics, antibacterial agents, antiviral agents, antiparasitic agents, antifungal agents, anaesthetics, analgesics, antiallergic agents, retinoids, free-radical scavengers, anti-pruriginous agents, kerato-lytic agents, anti-seborrhoeic agents, antihistamines, sulfides, immunosuppressant products and anti-proliferative products.

17. The composition according to claim 1, further comprising an additive chosen from the group of sequestrants, antioxidants, sunscreens, preserving agents, fillers, electrolytes, humectants, dyes, common mineral or organic acids or bases, fragrances, essential oils, cosmetic active agents, moisturizers, vitamins, essential fatty acids, sphingolipids, self-tanning compounds, calmatives and skin-protecting agents, pro-penetrating agents and gelling agents, or a mixture thereof.

18. The composition according to claim 15, wherein the composition contains from 0.1% to 2% by weight of the antagonist.

19. The composition according to claim 18, wherein the composition contains from about 0.75% to 1% by weight of the antagonist.
